(19) United States
(12) Patent Application Publication
(10) Pub. No.: US 2025/0225657 A1
(43) Pub. Date: Jul. 10, 2025

(54) METHODS OF PROCESSING OPTICAL IMAGES AND APPLICATIONS THEREOF

(71) Applicant: Apollo Medical Optics, Ltd., Taipei (TW)

(72) Inventors: I-Ling CHEN, Taipei (TW); Chih-Wei LU, Taipei (TW)

(21) Appl. No.: 18/427,059

(22) PCT Filed: Aug. 2, 2022
(86) PCT No.: PCT/US2022/039218

§ 371 Date: Jan. 30, 2024

Related U.S. Application Data
(60) Provisional application No. 63/228,580, filed on Aug. 2, 2021.

(51) Int. Cl. G06T 7/00; A61B 5/00; G06T 5/50; G06T 5/60; G06T 5/70; G06T 5/92; G06T 7/11; G06T 7/136

(52) U.S. Cl. CPC G06T 7/0014; A61B 5/0013; A61B 5/0022; A61B 5/0066; A61B 5/443; A61B 5/444; A61B 5/7203; A61B 5/7267; G06T 5/50; G06T 5/60; G06T 5/70; G06T 5/92; G06T 7/11; G06T 7/136; A61B 2576/02; G06T 2207/10101; G06T 2207/20081; G06T 2207/20084; G06T 2207/20216; G06T 2207/30088; G06T 2207/30196

(57) ABSTRACT

Provided herein is a method of segmenting features from an optical image of a skin comprising steps of receiving an optical image of a skin that contains at least one feature of an object: contrast-enhancing the feature's signals of the optical image from the background signals: segmenting the object in the enhanced optical image, and quantifying the feature from the optical image of the skin.

### BACKGROUND OF THE INVENTION

Traditionally, only histopathological sections have been used to visualize cellular changes in the skin. However, this gold standard method is invasive and not favored by patients with cosmetic concerns. In recent decades, an increasing number of non-invasive imaging tools, such as optical coherence tomography (OCT), reflectance confocal microscopy (RCM), and multiphoton microscopy, have become available to detect cellular changes in the skin with novel findings that might influence physicians' treatment decisions.

Non-invasive techniques, as described above, already detect pigmentary changes at a cellular level of resolution. The recently developed cellular resolution full-field optical coherence tomography (FF-OCT) device also allows real-time, non-invasive imaging of the superficial layers of the skin and provides an effective way to perform a digital skin biopsy of superficial skin diseases. Nevertheless, studies with quantitative measurements of the amount and intensity of pigment and analysis of its distribution in different skin layers remain scarce.

### SUMMARY OF THE INVENTION

The present invention relates to a method of segmenting features from an optical image of a skin, which is used to provide a novel way to label the features from the non-invasive optical images.

In one aspect, the present invention provides a method of processing optical image of a skin comprising
a) receiving an optical image of a skin that contains a feature of an object;
b) optionally performing a noise reduction to reduce the noise of the optical image;
c) contrast-enhancing the feature's signals of the object from the background signals;
d) segmenting the object in the optical image through at least one threshold value of the feature;
e) optionally categorizing the segmented object; and
f) quantifying the feature of said object from the optical image of the skin.

In another aspect provides a computer-aided system for skin condition diagnosis comprising an optical imager configured to provide an optical image of a skin; a processor coupled to the imager, a display coupled to the processor, and a storage coupled to the processor, the storage carrying program instructions which, when executed on the processor, cause it to carry out the method of disclosed herein.

Yet, in another aspect provides a method of identifying a pigment disorder comprising:
1) receiving an optical image of a suspected pigment disorder skin;
2) optionally performing a noise reduction to reduce the noise of the optical image;
3) contrast-enhancing the feature's signals of an object from the background signals wherein said object is melanin, melanosomes, melanocyte, melanophage, activated melanocyte, or combinations thereof;
4) segmenting the object in the enhanced optical image through at least one threshold value of the feature;
5) optionally categorizing the segmented object, wherein the feature is brightness or elongated structure;
6) quantifying the feature of said object from the optical image of the skin; and
7) identifying the suspected pigment disorder skin through the quantified value.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

A better understanding of the features and advantages of the present invention will be obtained by reference to the following detailed description that sets forth illustrative embodiments, in which the principles of the invention are used, and the accompanying drawings which:

FIG. 1A/B provide an exemplary block diagram illustrating how to categorize objects in an optical image from a skin (1A), and an exemplary block diagram illustration including further an optional noise reduction step and a computer-aided diagnosis step (1B).

FIG. 2 shows an exemplary noise reduction method by a deep learning architecture of the denoising convolutional neural network (DnCNN).

FIG. 3A/B show a series of the exemplary images (3A) to be processed by a denoised step to generate a low-speckle ground truth image (3B).

FIG. 4 shows a flowchart depicting the structure of the spatial compounding-based denoising convolutional neural networks (SC-DnCNN) trained for optical image denoising, such as the images from optical coherence tomography (OCT).

FIGS. 5A-F show a series of images illustrating an exemplary object categorization (i.e., melanin categorization) by the invention methods.

FIG. 6 provides an exemplary image with the labeled melanin after the object categorization.

FIG. 7A/B show the performance comparison of the exemplary OCT images (e.g., the perilesional skin images) without (7A) and with SC-DnCNN (7B) trained denoising step.

FIG. 8 is a block process diagram illustrating the method of categorizing activated melanocytes (dendritic cells).

FIG. 9 illustrates the result of the labeled activated melanocytes (dendritic cells) in the OCT image by the invention method disclosed herein.

### DETAILED DESCRIPTION OF THE INVENTION

Skin is the largest organ of the body. Skin contains three layers: the epidermis, the outermost layer of skin; the dermis, beneath the epidermis containing hair follicles and sweat; and a deeper subcutaneous tissue, which is made of fat and connective tissue. Melanocytes have dendrites that deliver melanosomes to the keratinocytes within the unit. The skin's color is created by melanocytes, which produce melanin pigment, and located in epidermis.

Skin pigmentation is accomplished by the production of melanin in specialized membrane-bound organelles termed melanosomes and by the transfer of these organelles from melanocytes to surrounding keratinocytes. Pigmentation disorders (or skin pigment disorders) are disturbances of human skin color, either loss or reduction, which may be related to loss of melanocytes or the inability of melanocytes to produce melanin or transport melanosomes correctly. Most pigmentation disorders involve the underproduction or overproduction of melanin. In some embodiments, a skin pigment disorder is albinism, melasma, or vitiligo.

In some pigment disorder diseases, such as melasma, the activated melanocyte has dendritic morphology; therefore, the activated melanocyte is also called the “dendritic cell”.

Dark skin-type individuals are prone to pigmentary disorders, such as melasma, solar lentigo, and freckles, among which melasma is especially refractory to treat and often recurs. The melanin amount in the skin is commonly used to monitor treatment response and classify patients with melasma. The existing melanin measurement tools are limited to skin surface detecting and cannot observe the distribution of melanin in the actual tissue structure.

In order to more precisely evaluate melanin related parameter of the skin and provide more specific treatments for each type of skin, detecting and identifying actual melanin features (such as content, density, area, or distribution) directly is needed.

Non-invasive techniques, including optical coherence tomography (OCT), reflectance confocal microscopy (RCM), and confocal optical coherence tomography, can be used to detect tissue changes (e.g., pigmentary changes) in the superficial layers of the skin at a cellular resolution to perform a digital skin biopsy of superficial skin diseases. In some embodiments, the tissue optical image is provided by an optical coherence tomography (OCT) device, a reflective confocal microscopy (RCM) device, a two-photon confocal microscopy device, an ultrasound imager, or the like. In certain embodiment, the optical image is provided through an OCT device or an RCM device. In certain embodiments, the tissue optical image is provided by an OCT device. With the non-invasive devices, such as an FF-OCT device, three-dimensional skin imaging can provide remarkable capabilities to visualize skin tissue structure and identify the critical features in skin layers, that can be used in assisting diagnosis of skin diseases and disorders. In some embodiments, the tissue optical image comprises epidermis slicing images. In some embodiments, the tissue optical image comprises a three-dimensional image (3D image), a cross sectional image (B-scan), or a vertical sectional image (E-scan). In certain embodiments, the tissue optical image is a B-scan image.

In some embodiments, the present invention provides a method of processing optical image of a skin, and applications therefrom enabling the detection (or identification) of skin diseases and/or disorders (such as a pigment disorder). The invention methods can be employed in a computer-aided system, which comprises an optical imager configured to provide an optical image of a skin; a processor coupled to the imager, a display coupled to the processor, and a storage coupled (i.e., in communication with) to the processor, the storage carrying program instructions which, when executed on the processor, cause it to carry out the method disclosed herein.

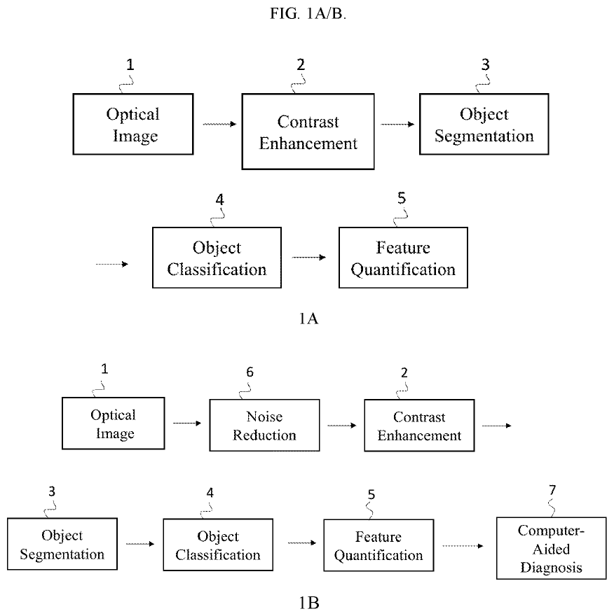

FIG. 1A provides an exemplary block diagram illustrating how to quantify a feature of objects in an optical image of a skin, comprising receiving an optical image of a skin comprising at least one feature of an object (i.e., a target of interest, such as melanin or activated melanocyte) (Step 1); contrast-enhancing the feature's signals of the object from the background signals (Step 2); segmenting the object in the enhanced optical image through at least one threshold value of the feature (Step 3), optionally categorizing (classifying) the segmented object (Step 4); and quantifying the feature of the segmented object from the optical image of the skin (Step 5). The feature, in some embodiments, is selected from the group consisting of brightness, particle area, particle size, particle shape, distribution position, and combinations thereof.

### Optional Noise Reduction Step

With a two-dimensional image sensor for parallel detection, and a low spatial coherence light source for illumination, a non-invasive device such as FF-OCT can acquire three-dimensional volumetric image with only one-dimensional mechanical scanning along axial direction. However, the image quality of cellular-resolution cross-sectional biological image may be suffered from the speckle noise because of the nature of coherent detection, even with low spatial coherence light source. Spatial compounding is a technique to reduce the speckle contrast significantly without much loss of resolution by averaging adjacent B-scans. For above mentioned cross-sectional imaging mode in FF-OCT devices that two-dimensional data acquired simultaneously and the B-scans are aligned inherently, the optional denoising image step base on spatial compounding can be realized without a pre-process of image registration. In some embodiments, the step comprises averaging the demodulated data in the thickness dimension which is close to 5 μm to approximate the typical thickness for H&E section. Since the sample structures from neighboring B-scan share some degree of correlation, the signal-to-noise ratio (SNR) can be improved by averaging, and the resultant image shows the average sample structure within a finite thickness.

Although common image filter (i.e. Gaussian, median filter) can be used to suppress the speckle noise, the drawback is loss of image detail, especially in case the image features of interest is of the similar dimension as the speckle grains.

In some embodiments, the denoising step comprises using a denoising neural network, such as spatial compounding-based denoising convolutional neural network (SC-DnCNN), which is trained with the compounded image data and can distinguish noises from signals while preserving the image details.

Spatial compounding (SC) is a commonly used technique to mitigate the speckle and Gaussian noise. The principle of SC is to induce changes in the speckle pattern between repeated measurements through the tiny position change of the subject. Then, these partially decorrelated multiple images measured from the sample are averaged to obtain low speckle images.

To train the denoising model, the noise maps are defined as the difference between before and after image averaging within a specific thickness. Through the powerful learning ability of deep convolutional neural network that can automatically extract multiple features as representations from the data, the trained SC-DnCNN model improves the image quality by noise prediction on single B-scan. In addition, the sampling thickness required to achieve spatial compounding can be reduced to increase the imaging speed.

FIG. 1B further illustrates certain embodiments of FIG. 1A comprising an optional noise reduction step (6), to reduce the noise of the optical image, and a computer-aided diagnosis step (7). In some embodiments, the noise of the optical image is reduced through a spatial compounding-based denoising convolutional neural network (SC-DnCNN), which provides effective noise reduction and improves image quality while maintaining the details of the optical image, especially OCT image.

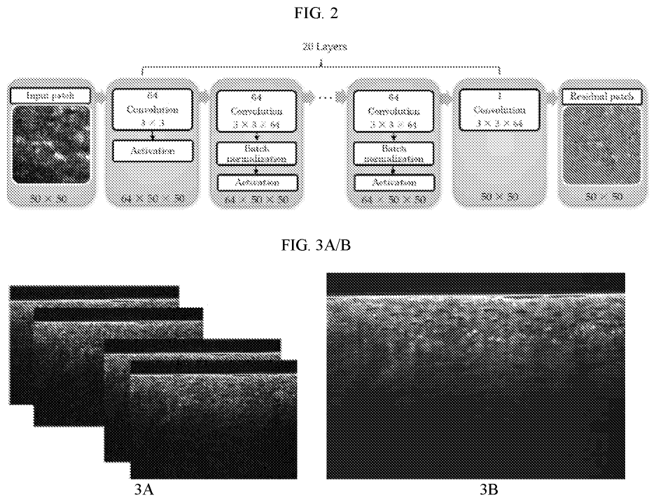

The SC-DnCNN is a pixel-wise noise prediction method that, in some embodiments, to be used to distinguish the noise in the signal, thereby improving the image quality. It follows the advantages of a denoising convolutional neural network (DnCNN), taking residual learning and batch normalization (BN) to speed up the training process and improve the denoising performance. As exemplary illustrated in FIG. 2, the deep architecture of a DnCNN is based on the concept of the visual geometry group (VGG) network and consists of multiple smaller convolutional layers. The composition of these layers can be divided into three main types. The first type appears in the first layer. It uses 64 filters with a size of 3×3 to generate 64 feature maps and then performs nonlinear conversion through rectified linear units (ReLU) on these feature maps as the input to the next layer. From the second layer to the penultimate layer, all these convolutional layers belong to the second type. Similarly, 64 filters with a size of 3×3×64 are used on the input maps, but unlike the previous layer, BN is added before ReLU. The BN is a normalization method that adjusts the distribution of input values to a normal distribution, which not only avoids the problem of gradient vanishing but also greatly accelerates the training speed. Finally, a filter with a size of 3×3×64 is used in the last layer as the output reconstruction.

In model training, the residual learning concept of deep residual network (ResNet) is applied to simplify the optimization process. The difference is that DnCNN does not add a shortcut connection between several layers, but directly changes the output of the network to a residual image. This means that the optimization goal of DnCNN is not the mean square error (MSE) between the real clean image and the network output, but the MSE between the real residual image and the network output. The residual image, the noise map, could be obtained by subtracting the clean image from the noisy image. The noise is randomly added to a clear image to simulate a noisy image. For example, regarding OCT images, the noise is mainly composed of the speckle noise, which multiplies the noise by the structure signal. Therefore, the ground truth is generated by using real OCT images rather than simulated ones.

Not limited to the exemplary method disclosed herein, SC-DnCNN is trained by a database containing noisy images and clean images, wherein the clean image is acquired by averaging N number of adjacent optical images, and the noisy image is acquired by averaging M number of adjacent optical images. N is greater than M. For example, N is 2 to 20, especially 5 to 15, especially 7 to 12. FIG. 3A/B show a series of the exemplary images (3A) processed by a denoised step with SC-based ground-truth generation to generate a low-speckle ground truth image (3B). 11-pixel lines are activated to acquire cross-sectional view (B-scan or cross-sectional scan) OCT images; accordingly, 11 adjacent virtual slices are generated for SC. The thickness of the compounding image is around 5 μm, which is close to histological slices. As the clean image, the composite image with low speckle is obtained by averaging 11 adjacent B-scans, which means N=11. In contrast, as a noisy image, the average image generated by compounding M pixel lines, where M<11.

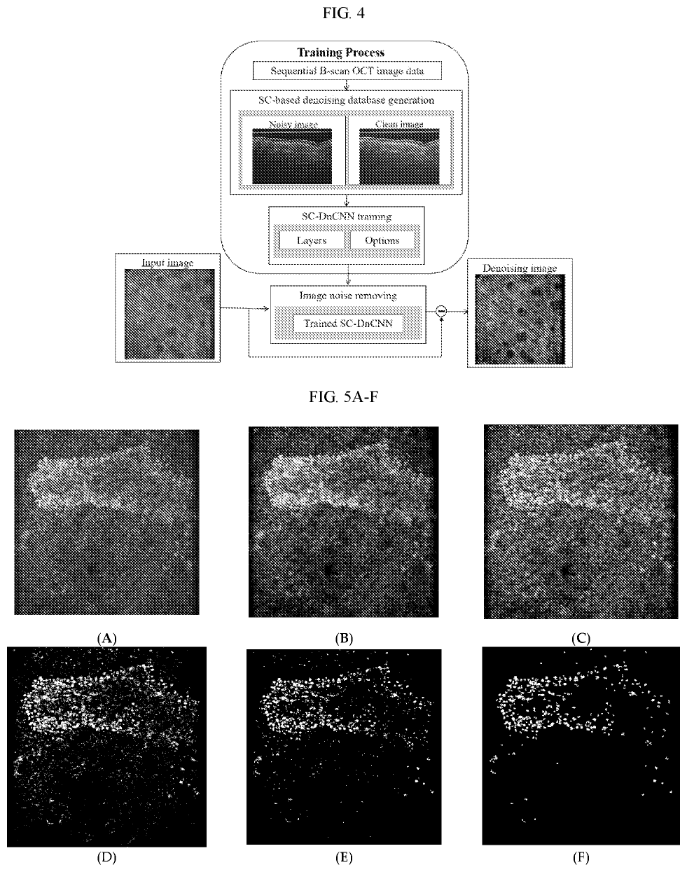

FIG. 4 shows the exemplary training and implementation structure of the SC-DnCNN model with the exemplary optical images. The training process of the model can be explained by an example provided as follows. A model trained with noisy images compounded by 5 pixel lines was chosen to improve the en-face scan (E-scan or horizontal scan) image quality in this example. To train the SC-DnCNN model, 512 paired patches with a size of 50×50 were randomly cropped in each pair of images (noisy image and noise map). The number of network layers to 20 was set the and used the stochastic gradient descent method to automatically learn the weights of the filter kernels. In this deep learning, the parameter settings for model training, including momentum, learning rate, mini-batch size, and epochs, were 0.9, 0.001, 128, and 50, respectively. In total, the model was trained and verified via 335 B-scan OCT images. The specifications of all B-scan data captured with the whole FF-OCT scan were 1024×715 pixels, about 0.5 μm/pixel image resolution, and storage in 8-bit pixel depth.

### Contrast Enhancement

To result more suitable optical images to identify the features in an object (i.e., a target of interest in the skin) and perform further image analysis, in some embodiments, some post-processing methods based on scanning depth and image brightness are used. First, the image correction is performed to compensate for the depth-dependent signal decay. The weights of image pixels can be set based on the distance from the skin surface to adjust the influence of the device (e.g., OCT) diffraction limit on the imaging depth in tissues.

In some embodiments, a contrast enhancement is applied, for example, by sharpening or brightening an image, to highlight key features. For instances, the contrast-limited adaptive histogram equalization was made. Different from ordinary histogram equalization, the advantage of the contrast-limited adaptive histogram equalization is to improve the local contrast and enhance the sharpness of the edges in each area of the image. Rather than using the contrast transform function on the entire image, this adaptive method operates several histograms on small regions in the image to redistribute the lightness values of the image. The neighboring areas are then combined using bilinear interpolation to eliminate artificially induced boundaries.

### Object Segmentation

Object segmentation is the process of partitioning an optical image into multiple image segments, also known as image regions or image objects (sets of pixels). For example, for extracting melanin (an object) related feature from background tissues in an OCT image, a binary image is created by segmenting the image in two parts (foreground and background) with a given brightness level b. By intensity thresholding, all pixels in the grayscale image with brightness greater than level b are replaced with the value 1, and other pixels are replaced with the value 0. The object segmentation process, in some embodiments, is handled by an algorithm for thresholding, clustering, and/or region growing that analyze the intensity, gradient, or texture, to produce a set of object regions.

In some embodiments, the object of the object segmentation step is melanin, melanosomes, melanocyte, melanophage, activated melanocyte (dendritic cell), or the combination thereof. In certain embodiments, the nonlimited feature is selected from the group consisting of a number, a distribution inside the skin, an occupied area in the skin, a size, a density, a brightness, a specific shape, and other optical signal features.

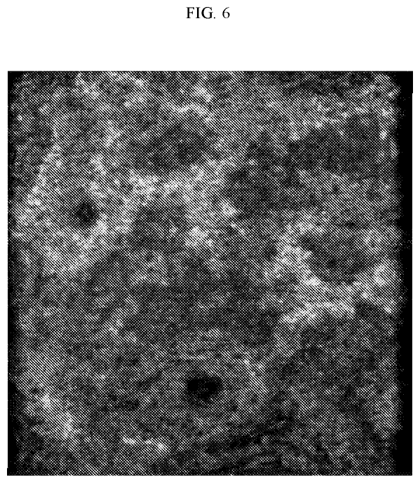

The E-scan OCT images are provided herein as an example to illustrate the process of segmenting an object (e.g., pigment related object) from the optical image of the skin of the present invention. As shown in FIG. 5A-F, first, an OCT E-scan image was provided (5A) containing a feature of melanin with hyper-reflective intensity compared with the surrounding tissues. Next, after reducing noise of the OCT image through SC-DnCNN, the image was shown in FIG. 5B. The feature's contrast was improved effectively. Then, a step of enhancing the melanin signals through contrast-limited adaptive histogram equalization (CLAHE) was used to stretch the contrast in each local area (approximately 12.5×12.5 μm²) to further enhance the feature of melanin whose intensity is stronger than the surrounding signal as shown in FIG. 5C. Several specified parameters in CLAHE, including the number of tiles into which the image is divided, the distribution type for creating the contrast transform function, and the limiting factor that controls the contrast enhancement effect, are determined through experiments to be 40×40, exponential (λ=0.1), and 0.001, respectively. Then next, a relatively loose brightness level with a threshold of 0.6 is given to filter out the target whose local signal does not reach a certain intensity as shown in FIG. 5D, which means that all pixels in the enhanced image that exceed the 153 gray level are regarded as candidates for melanin. Then next, binarizing the OCT image and extracting the melanin feature from the OCT image with a diameter greater than 0.5 mm was employed to produce the image shown in FIG. 5E, and the melanin feature with an area over 8.42 um² (about a circle with a diameter of 3.3 um) is shown in FIG. 5F. According to the granule size of aggregated melanin, the melanin is classified into two types: grain melanin (with diameter between 0.5˜3.3 um) and confetti melanin (with diameter >3.3 um). FIG. 6 shows a sample image of the labeled grain melanin and confetti melanin which may be labeled in different colors.

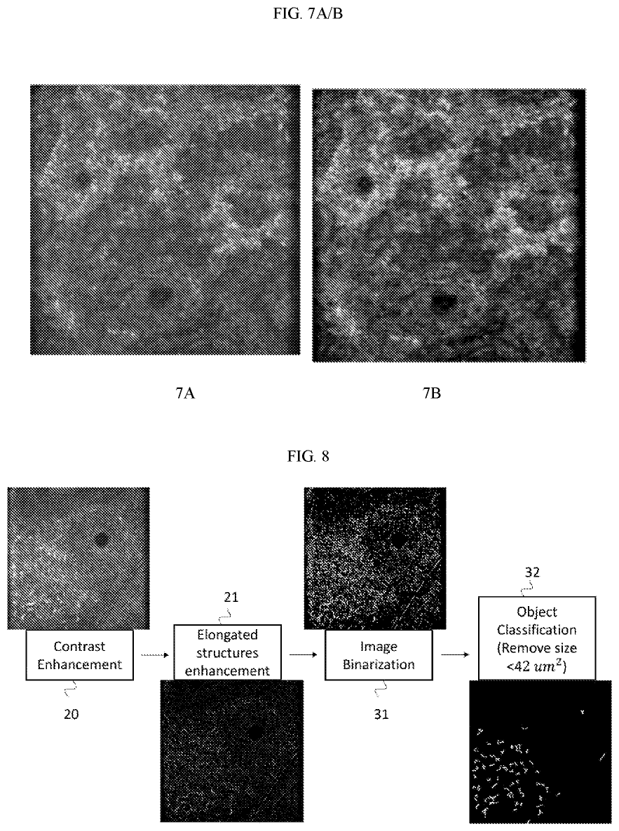

In accordance with the practice of the present invention, the denoising step is optional when the need arises. FIG. 7A/B show the performance comparison of the exemplary OCT images (e.g., the perilesional skin images) without (FIG. 7A) and with SC-DnCNN (FIG. 7B) trained denoising step. Comparing the results of the object segmentation process with and without SC-DnCNN, the OCT generated images processed by SC-DnCNN have apparently low speckle noise and high sharpness shown in FIG. 7B. These effects may help observe image details better and show obvious advantages for melanin recognition ability. It is particularly effective on the FF-OCT image processing.

### Feature Quantification

Feature quantification provides an effective way for physicians to monitor skin diseases or disorders (e.g., the pigment disorders).

By way of illustration, the features of melanin are quantified. In certain embodiments, the melanin related parameters (features) are listed in Table 1 where the feature quantification is based upon. Images acquired from E-scan are used as an example to describe the complete image processing flow of melanin feature quantization. For B-scan and C-scan, the methods and steps of image processing and analysis can be adjusted reasonably and flexibly based on the data under the same concept.

TABLE 1
Quantitative features of melanin-related parameters (features) on E-scan OCT images.

| Form | Category | Feature | Definition |
|---|---|---|---|
| All | | G_density | The density of the melanin in the tissue |
| melanin | | | |
| grain | Area | G_area | The area of all grain melanin |
| | Distribution | G_density | The density of the grain melanin in the tissue |
| | Brightness | G_intensity_min | The minimum brightness of the grain melanin |
| | | G_intensity_max | The maximum brightness of the grain melanin |
| | | G_intensity_mean | The average brightness of the grain melanin |
| | | G_intensity_SD | The standard deviation of the grain melanin brightness |
| confetti | Area | C_area | The area of all confetti melanin |
| | Distribution | C_distance_mean | The average distance between the centroid of confetti melanin |
| | | C_distance_SD | The standard deviation of the distance of confetti melanin |
| | | | centroid |
| | Shape | C_roundness | The average roundness of all confetti melanin |
| | | C_size_min | The minimum size of all confetti melanin |
| | | C_size_max | The maximum size of all confetti melanin |
| | | C_size_mean | The average size of all confetti melanin |
| | | C_size_SD | The standard deviation of the confetti melanin size |
| | Brightness | C_intensity_min | The minimum brightness of the confetti melanin |
| | | C_intensity_max | The maximum brightness of the confetti melanin |
| | | C_intensity_mean | The average brightness of the confetti melanin |
| | | C_intensity_SD | The standard deviation of the confetti melanin brightness |

In an example of feature quantification processing provided herein, 96 lesion images and 48 perilesional skin images that contained three layers: the en-face stratum spinosum, the dermal-epidermal junction (DEJ), and papillary dermis were used. Melanin is segmented as described herewith. As to the melanin feature quantification, the quantitative features extracted from the segmented melanin are classified into two groups: grain and confetti melanin in accordance with the practice of the present invention. Per Table 1, the area-based features separately count the total area of all grain melanin and confetti melanin segmented from an optical image. The distribution-based feature of all grain melanin, G_density, is based on the total area of the tissue in the image to calculate the proportion of its area, where the tissue is defined as the signal whose grayscale value is greater than 38 in the enhanced image. The distribution-based features of all confetti melanin are related to their distance in two-dimensional space. C_distance_mean and C_distance_SD, respectively, use the centroid of each confetti melanin to compute the average and standard deviation of the distance between each other. In addition, the features based on shape and brightness, respectively, provide statistical information to determine the size and intensity of all melanin in the image. To extract the C_roundness feature, a simple metric indicating the roundness of confetti melanin is defined as

$$roundness = \frac{4 π * area}{{perimeter}^{2}} \qquad ( 1 )$$

To explore the correlation between melasma and melanin, the potential of quantitative features in distinguishing lesion images from perilesional skin images was evaluated by several statistical hypothesis tests. For comparison, all data before and after the image denoising were also tested to observe the effect of the SC-DnCNN model under the method of the present invention. Whether there was a normal distribution of a feature was determined by the Kolmogorov-Smirnov test. Subsequently, the difference of each feature between the lesion and perilesional skin cases was evaluated with the mean+SD in a normal distribution and the median in a non-normal distribution by using Student's t-test and the Mann-Whitney U-test, respectively. With the significance analysis, a p-value of less than 0.05 indicated the difference was significant.

Tables 2 to 3 list the performance difference under the method disclosed herein performed with and without SC-DnCNN. The p-values and mean+SD of all distinct features generated before and after image denoising were extracted and analyzed. Table 2 shows that the C_distance_mean, a feature representing the average distance of each centroid of all confetti melanin, differs markedly between lesions and perilesional skin (p=0.0402) with denoising. The average distances of confetti melanin in perilesional skin and lesion images were 200 um and 193.5 um, respectively, while they were 206.1 um and 200.3 um, respectively, for the method without SC-DnCNN. The value of the C_distance_mean in the lesion image tended to be smaller than that of the perilesional skin image. However, the difference was not statistically significant (p=0.0502) when image denoising was not performed.

TABLE 2
The p-values and mean ± SD of the significant features
were used to identify lesions in the denoising images.

| | | Perilesional skin | Lesion | |
|---|---|---|---|---|
| Feature | Image denoising | (mean ± SD) | (mean ± SD) | p-value |
| C_distance_mean | Before | 206.1 ± 17.4 | 200.3 ± 14.0 | 0.0502 |
| | After | 200.0 ± 18.5 | 193.5 ± 15.9 | 0.0402* |

*p-value < 0.05 shows the statistically significant difference.

TABLE 3
The p-values and mean ± SD of the significant features
used to identify lesions in the subset without the SC-DnCNN.

| | | Image | Perilesional skin | Lesion | |
|---|---|---|---|---|---|
| Layer | Feature | denoising | (mean ± SD) | (mean ± SD) | p-value |
| Stratum spinosum | C_distance_mean | Before | 206.4 ± 12.9 | 194.3 ± 11.4 | 0.0036* |
| | | After | 198.1 ± 12.9 | 185.3 ± 13.3 | 0.0032* |
| | C_distance_SD | Before | 103.7 ± 6.8 | 98.8 ± 6.0 | 0.0202* |
| | | After | 101.1 ± 7.4 | 96.2 ± 6.4 | 0.0312* |
| Dermal-epidermal | All_density | Before | 5.343 ± 1.123 | 5.865 ± 1.124 | 0.1393 |
| junction | | After | 4.905 ± 0.851 | 5.484 ± 0.984 | 0.0426* |

*p-value < 0.05 shows the statistically significant difference.

Besides this, the dataset was divided into three subsets according to the skin layer (stratum spinosum, DEJ, and papillary dermis), and evaluated the difference between the melanin features that could distinguish lesions in each subset. The p-values and mean±SD of different features generated before and after image denoising for each subset are also summarized in Table 3. In the stratum spinosum, both significant features symbolize the distribution of the confetti melanin, where the larger the C_distance_mean is, the more dispersed the melanin will be. Plus, the smaller the C_distance_SD is, the more evenly distributed the melanin in the entire image will be. That means that compared with the perilesional skin, the distribution of confetti melanin in the lesion is more clustered in the local area of the image. The p-values of C_distance_mean and C_distance_SD were 0.0036 and 0.0202, respectively, before image denoising; while they were 0.0032 and 0.0312, respectively, after image denoising. Without executing the image denoising step, all the quantitative features of the DEJ and papillary dermis were not significantly different between the lesion and the perilesional skin. Through SC-DnCNN, the p-value of all_density in the DEJ was reduced from 0.1393 to 0.0426. For the lesion images, it indicates that the feature of the grain melanin density tends to be higher than that in the perilesional skin image.

Based on the above results, it is applicable to quantitatively evaluate and compare some melanin characteristics belonging to the lesion, including its appearance in different skin layers. When observing the OCT images within a lesion, the confetti melanin appears dense and concentrated in the stratum spinosum, while the grain melanin has a higher density in the DEJ. Different skin layers produce different forms of melanin, and their appearance on OCT images is also different.

In certain embodiments provides a method of identifying a pigment disorder of a skin comprising receiving an optical image of a suspected pigment disorder skin; optionally performing a noise reduction to reduce the noise of the optical image; contrast-enhancing the feature's signals of an object from the background signals wherein said object is melanin, melanosomes, melanocyte, melanophage, activated melanocyte, or combinations thereof; segmenting the object in the enhanced optical image through at least one threshold value of the feature; categorizing the segmented object; quantifying the feature of said object from the optical image of the skin; and identifying the suspected pigment disorder skin through the quantified value.

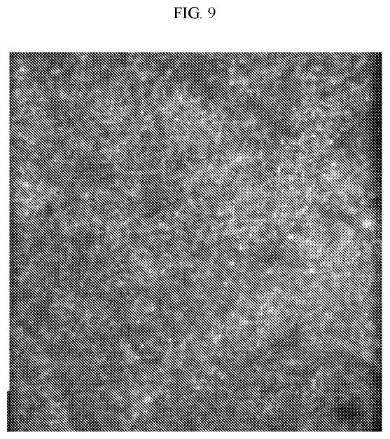

In some embodiments yet provides another example of an object segmentation, wherein the object is activated melanocytes. As reported previously, UV or laser treatment will activate melanocyte forming dendrite feature to secrete out melanin to epidermis layer to protect skin damage. For this reason, the dendrite morphology melanocytes are called “dendritic cells”. The general steps for segmenting the dendritic cells are the same as shown in FIG. 1A. FIG. 8 shows a block process diagram (with the exemplary images in each step) illustrating the method of categorizing/classifying activated melanocytes (dendritic cells) from the optical images (e.g., the exemplary OCT images). The contrast enhancing step further comprises features related to various morphology of dendritic cells such as an elongated structures enhancement. After providing OCT images, which are acquired by averaging 5 to 10 adjacent optical images through spatial compounding process; the contrast of the dendritic cell of the OCT image is enhanced (20); next, the feature of elongated structure of dendritic cells is enhanced by e.g., Hessian based Frangi Vessel filter (21). During the object segmentation step, the enhanced optical image is converted to a binary image (31) by thresholding to make the image easier to analyze. Then the dendritic cell is classified (32) for recognition, particle size <42 um²; and subsequently the classification of the dendritic cells is labelled as shown in FIG. 9.

In accordance with the practice of the present invention, quantifications of the segmented dendritic cells are also realized based on the features listed in table 4.

TABLE 4
Dendritic cells related parameters.

| Form | Category | Feature | Definition |
|---|---|---|---|
| DC | Quantity | Amount | Total number of DCs |
| | | Area | Total area of DCs |
| | | Size_Min | Size of the smallest DC |
| | | Size_Max | Size of the largest DC |
| | | Size_Mean | Average size of all DCs |
| | | Size_std | Size variation of all DCs |
| | Shape | Irregularity | Average irregularity of all DCs |
| | | Aspect ratio | Average aspect ratio of DCs |
| | | Roundness | Average roundness of all DCs |
| | | Length | Average length of DCs |
| | | Width | Average width of DCs |
| | Distribution | Density_Mean | Average distance between the centroids of DCs |
| | | Density_std | Variation in the distance between the centroids of DCs |
| | Brightness | Intensity_Min | Minimum brightness of DCs |
| | | Intensity_Max | Maximum brightness of DCs |
| | | Intensity_Mean | Average brightness of all DCs |
| | | Intensity_Std | Brightness variation of DCs |

In some embodiments provide a method of processing optical image of a skin comprising: a. receiving an optical image of a skin that contains a feature of an object; b. optionally performing a noise reduction to reduce the noise of the optical image; c. contrast-enhancing the feature's signals of the object from the background signals; d. segmenting the object in the enhanced optical image through at least one threshold value of the feature; e. optionally categorizing the segmented object; and f. quantifying the feature of said object from the optical image of the skin. In some embodiments, the method further comprises a computer-aided diagnosis step after the step of feature quantification. In some embodiments, the optical image is an optical coherence tomography (OCT) image, a reflectance confocal microscopy (RCM) image, or a confocal optical coherence tomography image. In some embodiments, the optional noise reduction step reduces the noise of the optical image through a spatial compounding-based denoising convolutional neural network (SC-DnCNN). In certain embodiments, the SC-DnCNN is trained to distinguish the noise of the optical image. In certain embodiments, the SC-DnCNN is trained by a database containing noisy images and clean images. In certain embodiments, the clean image is acquired by averaging N number of adjacent optical images, the noisy image is acquired by averaging M number of adjacent optical images, and N is greater than M. In some embodiments, the object is melanin, melanosomes, melanocyte, melanophage, activated melanocyte, or combinations thereof. In certain embodiments, the object is melanin, melanocyte, or activated melanocyte. In certain embodiments, the object is melanin. In certain embodiments, the feature is brightness, particle area, particle size, particle shape, or distribution position in the skin. In certain embodiments, the feature is brightness and/or particle shape (e.g., an elongated structure). In some embodiments, the optical image is acquired by averaging at least two adjacent optical images. In certain embodiments, Step e comprises categorizing the object to grain melanin, or confetti melanin. In some embodiments, the contrast enhancement step is applied by sharpening or brightening the optical image to highlight a feature of said object. In some embodiments, the object segmentation step is handled by an algorithm for thresholding, clustering, and/or region growing, that analyzes intensity, gradient, or texture to produce a set of object regions.

In some embodiments provides a computer-aided system for skin condition (e.g., skin diseases or disorders such as skin pigment disorder) diagnosis, which comprises an optical imager configured to provide an optical image of a skin; a processor (such as a computer) coupled to the imager, a display coupled to the processor, and a storage coupled to the processor, the storage carrying program instructions which, when executed on the processor, cause it to carry out the invention method disclosed herein in the computer program (e.g., See FIG. 1B). In some embodiments, the imager is an optical coherence tomography (OCT) device, a reflectance confocal microscopy (RCM) device, a confocal optical coherence tomography device, or the like. In certain embodiments, the imager is an optical coherence tomography (OCT) device.

In some embodiments, the system, network, method, and media disclosed herein include at least one computer program, or use of the same. A computer program includes a sequence of instructions, executable in the digital processing device's CPU, written to perform a specified task which may refer to any suitable algorithm. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

In some embodiments, computer systems or cloud computing services are connected to the cloud through network links and network adapters. In an embodiment, the computer systems are implemented as various computing devices, for example servers, desktops, laptops, tablet, smartphones, Internet of Things (IoT) devices, and consumer electronics. In an embodiment, the computer systems are implemented in or as a part of other systems.

Owing to its capability of reaching real-time and stable detection results, together with its objectivity and precision when describing melanin features, this method could surely represent an attractive tool to address pigment classification problems with such requirements.

Although preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1. A method of processing optical image of a skin comprising
a) receiving an optical image of a skin that contains a feature of an object;
b) optionally performing a noise reduction to reduce the noise of the optical image;
c) contrast-enhancing the feature's signals of the object from background signals;
d) segmenting the object in the enhanced optical image through at least one threshold value of the feature;
e) optionally categorizing the segmented object; and
f) quantifying the feature of said object from the optical image of the skin.

2. The method of claim 1, further comprising a step of computer-aided diagnosis after step e.

3. The method of claim 1, wherein step b reduces the noise of the optical image through a spatial compounding-based denoising convolutional neural network (SC-DnCNN).

4. The method of claim 3, wherein the SC-DnCNN is trained to distinguish the noise of the optical image.

5. The method of claim 4, wherein the SC-DnCNN is trained by a database containing noisy images and clean images.

6. The method of claim 5, wherein the clean image is acquired by averaging N number of adjacent optical images, the noisy image is acquired by averaging M number of adjacent optical images, and N is greater than M.

7. The method of claim 1, wherein the object is melanin, melanosomes, melanocyte, melanophage, activated melanocyte, or combinations thereof.

8. The method of claim 7, wherein the feature is brightness, particle area, particle size, particle shape, or distribution position in the skin.

9. The method of claim 8, wherein the feature is brightness.

10. The method of claim 1, wherein the optical image is acquired by averaging at least two adjacent optical images.

11. The method of claim 7, wherein the object is melanin, melanocyte, or activated melanocyte.

12. The method of claim 11, wherein the object is melanin.

13. The method of claim 12, wherein Step e comprises categorizing the object to a grain melanin, or confetti melanin.

14. The method of claim 1, wherein the optical image is an optical coherence tomography (OCT) image, a reflectance confocal microscopy (RCM) image, or a confocal optical coherence tomography image.

15. A computer-aided system for skin condition diagnosis comprising an optical imager configured to provide an optical image of a skin; a processor coupled to the imager, a display coupled to the processor configured to output the diagnosis, and a storage coupled to the processor, the storage carrying program instructions which, when executed on the processor, cause it to carry out the method of claim 2.

16. The computer-aided system of claim 15, wherein the imager is an optical coherence tomography (OCT) device, a reflectance confocal microscopy (RCM) device, or a confocal optical coherence tomography device.

17. (canceled)

18. The computer-aided system of claim 15, wherein the storage comprises a cloud based storage.

19. (canceled)

20. The computer-aided system of claim 15, wherein the skin condition is a skin cancer, or a skin pigment disorder.

21. The computer-aided system of claim 20, wherein the pigment disorder is albinism, melasma, or vitiligo.

22. (canceled)

23. A method of identifying a pigment disorder of a skin comprising
1) receiving an optical image of a suspected pigment disorder skin;
2) optionally performing a noise reduction to reduce the noise of the optical image;
3) contrast-enhancing the feature's signals of an object from the background signals wherein said object is melanin, melanosomes, melanocyte, melanophage, activated melanocyte, or combinations thereof;
4) segmenting the object in the enhanced optical image through at least one threshold value of the feature;
5) quantifying the feature of the object from the optical image of the skin; and
6) identifying the suspected pigment disorder skin through the quantified value.
